(19) United States
(12) Patent Application Publication
(10) Pub. No.: US 2004/0028666 A1
(43) Pub. Date: Feb. 12, 2004

(54) Enhanced flavour production in or relating to food by cultivation of various food-grade micro-organisms

(76) Inventors: Gerrit Smit, Ede (NL); Eman Hussen El Sayed Ayad, Ede (NL)

Correspondence Address: Charles A Muserlian, Bierman Muserlian & Lucas, 600 Third Avenue, New York, NY, 10016, US

(21) Appl. No.: 10/312,651

(22) PCT Filed: Jul. 1, 2001
(86) PCT No.: PCT/EP01/07558

(30) Foreign Application Priority Data
Jun. 30, 2000 (EP) 002022762

(51) Int. Cl. C12N 1/20

(52) U.S. Cl. 424/93.45; 435/252.4

(57) ABSTRACT

New mixed cultures of two or more micro-organism strains are provided wherein at least one of said micro-organism strains which are comprised in said mixed culture is individually selected on the basis of its ability to perform part of an enzymatic pathway, and said two or more selected micro-organism strains together form a complete pathway towards a desired flavour component. Preferably, the mixed culture is a culture for the production of a fermented product, such as yogurt or cheese or sausage. Said two or more micro-organism strains are preferably co-cultivated. Particular and preferred embodiments are starter cultures for the manufacturing of cheese, comprising a combination of various Lactoccocus strains and a combination of a Brevibacterium strain and a Staphylococcus strain, respectively.

### TECHNICAL FIELD

The present Invention generally relates to the field of flavour production, in particular in or relating to food, and fermented food, such as cheese, yogurt, and sausages. More in particular, the invention relates to methods and means for enhanced cheese flavour production by the cultivation of various food-grade micro-organisms, such as lactic acid bacteria, as starter cultures.

### BACKGROUND AND PRIOR ART

Microbial flavour development is essentially an enzymatic process performed by micro-organisms, and plants. Various micro-organisms such as fungi, yeasts and bacteria have been identified and selected for their special flavour production (c.f. R. G. Berger, 1992, in: Bioformation of flavours pp. 21-32, eds. R. L. S. Patterson, B. V. Charlwood, G. MacLeod, and A. A. Williams, Royal Soc. Chem., UK.). These flavours arise from the ability of micro-organisms to convert a component or substrate in the growth medium through a series of enzymatic steps into one or more specific flavour compounds. The commercial production of microbially-produced flavours usually takes place by fermentation, or by growing in situ in or on foodstuff like dairy food or sausages. For instance, during cheese ripening, proteolytic enzymes of the starter culture play a significant role in protein breakdown (Law et al. 1974; Bie and Sjostrom 1975a; Bie and Sjostrom 1975b). This breakdown of proteins is important for the formation of a desirable flavour and texture, and therefore proteolysis has been investigated extensively (Pritchard and Coolbear 1993; Visser 1993; Exterkate and Alting 1995; Exterkate et al. 1995; Law and Mulholland 1995). It has been demonstrated that proteinases and peptidases of starter bacteria release peptides and free amino acids from casein (Olson 1990; Visser 1993; Engels and Visser 1994).

The relationship between release of amino acids and flavour formation in cheese has been assumed for a long time (Mulder 1952; Solms 1969). Amino acids may contribute to flavour either directly or indirectly by serving as precursors of volatile aroma compounds such as aldehydes, acids, alcohols, esters and sulphur compounds (Engels and Visser 1996). In recent years, it has become clear that the conversion of amino acids Into volatile (flavour) compounds plays an important role in the ripening process leading to flavour development. A number of enzymes involved in amino acid conversion have been identified in various starter cultures (Schmidt and Lenoir 1974; Nakazawa et al. 1977; Lee and Richard 1984; Lee et al. 1985; Alting et al. 1995; Yvon et al. 1997; Yvon et al. 1998). Generally, these enzymes are involved in various types of reactions, including deamination, transamination, decarboxylation and cleavage of the amino acid side chains. A survey of some general pathways of the breakdown of amino acids is disclosed by Hemme et al. 1982, which was reproduced by Engels 1997 who also presented the following table with documented examples of amino acid-derived cheese volatiles.

TABLE 1
Examples of products formed by breakdown of amino acids during cheese
ripening

| Amino acid | Volatile product | Flavour |
|---|---|---|
| Leu | 3-methyl-1-butanol | fresh cheese, fruity |
| Ile | 2-methylbutanal | malty, harsh |
| Met | methanethiol | onion, cheese |
| Phe | phenylacetaldehyde | rose |
| Tyr | phenol | phenol, medicinal |
| Thr | acetaldehyde | “green”, yogurt |
| Val | 2-methylpropanal | malty, harsh |

Lactic acid bacteria (“LAB”) which are present in all types of cheeses, play a major role in generating flavour compounds from amino adds. In lactococci, transamination is a first step in the conversion of aromatic and branched-chain amino acids, since no oxidative deamination or decarboxylation was detected in several strains of Lactococcus lactis subsp. lactis or cremoris (Thirouin et al. 1995; Gao et al. 1997; Yvon et al. 1997; Engels 1997; Engels et al. 2000). Recently, a number of transaminases in LAB has been identified and characterized (Engels 1997; Yvon et al. 1997; Roudot-Algaron and Yvon 1998; Gao and Steele 1998; Rijnen et al. 1999; Yvon et al. 1997). The α-keto acids produced by transamination of the amino acids can either undergo spontaneous dégradation (Gao et al. 1997) or are degraded enzymatically into the corresponding aldehydes or carboxylic acids (Thirouin et al. 1995; Smit et al. 2000). The transamination reaction is catalysed by aminotransferases, which transfer the α-amino group of amino acids to an α-keto acid acceptor.

In addition to single starter cultures, it is common practice to use mixed starter 25 cultures in cheese manufacture. For example, mixed starter cultures of Lactococcus species are normally used for making of semi- and hard type of cheeses (e.g., Gouda, Cheddar, Tilsiter, Saint Paulin); Lactococcus with Propionibacteria (and L. bulgaricus) for Maasdammer type of cheese (e.g., Leerdam); Lactococcus species with L. helveticus and S. thermophilus for the preparation of Proosdij-type of cheeses (e.g., Old Amsterdam, Cantenaar, Milner); Lactobacillus helveticus, L. acidophilus and Streptococcus thermophilus strains for making hard type of cheeses (e.g., Parmesan, Manchevo); same cultures with Propionibacterium for making Emmentaler and Gruyère cheeses. Furthermore, for cheeses with surface ripening, additional cultures are used such as, for example, Penicillium camemberti (e.g., Brie); P. roquefortii (e.g., Roquefort); and Brevibacterium, Debaromyces, Staphylococcus, Arthrobacter, and Corynebacterium species for red-smear cheeses (Saint Paulin, Kernhem kaas, Tilsiter, etc.). In such mixed cultures various interactions may occur, which not only affect the composition of the mixtures but also may have an impact on flavour formation. Depending on the enzymes present in the cultures, different flavours can develop due to the contribution of many enzymes of these cultures which lead to final flavour.

Although it is common practice in cheese-making (as well as in other fermentations) to use mixtures of cultures, these cultures have not been selected sofar for enhancing the total metabolic pathway for the formation of volatile flavour components. The cultures were indeed combined predominantly for reasons such as preventing or reducing sensitivity for phage attack (common practice in Gouda cheese making) and formation of eyes in the cheese (e.g., Maasdammer, Gruyère). In the case of cheeses with so-called flavour-forming strains (e.g., Camembert, Proosdij cheese, red-smear cheeses), the flavour-forming adjunct cultures are selected on their ability to form a specific flavour, and not on the basis of complementing a pathway together with the other cultures used, which is the purpose of the present invention.

DE 199 03 538 describes a concentrated aqueous solution of metabolic products from water kefir micro-organisms having a neutral taste and improved storage characteristics. Kefir micro-organisms comprise a variety of symbiotic micro-organisms which are difficult to separate, inter alia from Lactobacillus and yeast cultures, which grow together as particles. This reference does not disclose or suggest a product from a culture of mixed but defined micro-organisms with improved flavour properties resulting from a complemented metabolic pathway by individual strains from said culture.

EP 0 359 295 disdoses a method for preparing cheese using both a mesophilic starter containing lactic acid bacteria exhibiting an optimal growth at below 33° C., and a culture of thermophilic lactic acid bacteria which is APS₁₃. The use of APS₁₃ resulted in a characteristic flavour which was not obtained with the mesophilic culture alone. Moreover, in this manner the technology of semi-hard cheese making is not affected. Whereas this reference teaches the production of a new broad flavour profile, the present invention is directed to the enhancement of a more specific taste by increasing or adding certain flavour components to the flavour palette due to specific interaction between strains in the starter culture.

EP 0 521 331 discloses a soy milk fermentation process in which the seed of micro-organisms comprises at least two different lactic acid producing bacterial strains, one of which being Lactococcus lactis subsp. lactis var. diacetylactis, for preparing yogurt-like food. No teaching is provided how to select micro-organisms for enhancing taste characteristics in mixed cultures.

L. Lesage-Meesen et al., J. Biotechnol. 50:107-113 (1996) and J. Sci Food Agric 79:487-490, describe a two-step bioconversion process for vanillin production from ferulic acid (enzymatically isolated from sugar beet pulp) combining Aspergillus niger and Pycnoporus cinnabarinus. In a first step, natural ferulic acid was metablised by A. niger into vanillic acid which was then converted to vanillin by P. cinnabarinus in a yield of 100 mg/l. These references neither disclose nor suggest mixed cultures which form the basis of the present invention.

Many starter cultures for dairy products have been tried or have been used commercially throughout the years and various studies were made to the role of starter cultures in flavour formation, the enzymes involved in the conversion of amino acids, and the regulation of enzymatic conversions to control formation of flavour compounds during ripening. Genetic approaches have been studied for some years to enhance the flavour formation. We have now surprisingly found that combining actions of two or more micro-organisms to the extent that they form together a metabolic pathway to the formation of certain desired compounds, in particular certain flavour compounds, is an excellent way for enhancing flavour production.

### SUMMARY OF THE INVENTION

It is an object of the present invention to provide a mixed culture of two or more microorganism strains, wherein at least part of said micro-organism strains comprised in said mixed culture is individually selected on the basis of its ability to perform part of an enzymatic pathway, and said individually selected micro-organism strains together form a completed pathway towards a desired flavour compound.

It is another object of the invention to provid new starter cultures for use in the manufacturing of fermented (e.g. dairy) products with enhanced or tailor-made flavour formation, comprising a combination of two or more micro-organism strains which are individually less capable or incapable of converting amino acids into volatile compounds to the extent of forming the desired flavour but when used together convert amino adds into volatile compounds under appropriate conditions giving the desired flavour. Said two or more micro-organism strains are preferably co-cultivated. If desired, the micro-organism strains of choice can also be cultivated consecutively to reach the same or a similar effect, but this embodiment is less preferred.

A particular and preferred embodiment of the invention is a starter culture for the manufacturing of cheese which comprises a combination of two Lactococcus strains, in particular Lactococcus cremoris strain B1157 and Lactococcus cremoris strain SK110. Another particular embodiment of the invention is a starter culture for the manufacturing of cheese which comprises a combination of a Brevibacterium strain and a Staphylococcus strain, in particular Brevibacterium casei strain B1392 en Staphylococcus saprophyticus strain B1144.

The mixed cultures according to the present invention are suitably used for the production of a variety of products including, for example, foodstuff, food ingredients, and flavours. In a preferred embodiment the mixed cultures are used as a starter culture in the manufacturing of dairy products, in particular cheese and yogurt, most preferably cheese.

These and other objects and embodiments of the present invention will be described in more detail in the description which follows.

### BRIEF DESCRIPTION OF THE DRAWINGS

FIG. 1. Changes in starter populations in milk cultures prepared with strains, B1157 and B851 (open bars) and strain SK110 (filled bars). A, strain B1157; A₁(2:1), A₂ (1:2), strain B1157: strain SK110 and B, strain B851; B₁(2:1), B₂ (1:2), strain B851:strain SK110, represent viable counts in milk cultures (mean of duplicates).

FIG. 2. Relative amounts of branched-chain aldehydes 2-methylpropanal (“2MeA3”), 2-methylbutanal (“2MeA4”) and 3-methylbutanal (“3MeA4”) formed during incubation of individual and combined strains in milk culture.

FIG. 3. Relative amounts of branched-chain aldehydes formed by B1157, SK110 and (B1157+SK110 2:1) strains in milk cultures without no additives (A); with: leucine (B); isoleucine (C); valine (D); α-keto isocaproic acid (“KICA”) (E); α-keto-β-methyl-n-valeric acid (F) and α-keto-isovaleric acid (G).

FIG. 4. Relative amount of branched-chain amino acids (Leu, Ile and Val) in milk cultures. Milk (blank), and milk cultures incubated with SK110, B1157, (B1157+SK110 2:1), (B1157+SK 110 1:1) and (B1157+SK110 1:2).

FIG. 5. Proposed pathway of leucine by enzymes from individual and mixed lactococcal starter cultures B1157, B851 and SK110. (A) General pathway for the breakdown of casein; (B) SK110; (C) B1157; (D) defined culture (B1157+SK110); (E) B851; and (F) mixed culture (B851+SK110). In the decarboxylation step, a narrow arrow represents low decarboxylase activity whereas a thick arrow represents high decarboxylase activity.

### DETAILED DESCRIPTION OF THE INVENTION

As explained in the introduction, microbial flavour production is the result of a micro-organism's ability to utilize components in the growth medium/solid substrate and to convert this substrate in a series of—mostly enzymatic—processes into one or more flavour compounds.

Strains have been selected for their capability to perform the complete sequence of releasing the substrate from the medium and the various conversion steps. The total output of flavour formation was the result of the combined enzymatic activities that the micro-organism demonstrated. For example, amino acids play an important role in the development of cheese flavour. Most of the free amino acids are liberated by the hydrolysis of caseins from milk by proteolytic enzymes. The direct role of amino acids for cheese flavour is, however, limited. Catabolism of amino acids during cheese ripening as a source of flavour compounds has been frequently suggested (for a survey, see Engels 1997). The amino acids are exposed to enzymic but probably also to chemical reactions. Micro-organisms present in the cheese and/or added during the manufacturing of the cheese cause a variety of reactions, such as proteolyse, transamination and decarboxylation whereby amino acids eventually are degraded to volatile compounds, each having a specific flavour.

Various technologies have been applied to enhance the flavour production of micro-organisms. These include, for example, the addition of enzymes to the growth medium to enhance the release of substrates from the growth medium, the adaptation of fermentation conditions, and genetic approaches to enhance one or more of the enzymatic steps involved in flavour formation.

The present invention provides, in one aspect, mixing cultures of two or more micro-organism strains for the production of a wide range of products wherein at least part of said micro-organism strains which are comprised in said mixed culture is individually selected on the basis of its ability to perform part of an enzymatic pathway, and said individually selected micro-organism strains together form a completed pathway towards a desired flavour or flavour component. It has been found that this approach results in a higher production of desired flavour compounds or mixtures, as well as in an easier way of selecting strains.

As mentioned above, prior art cultures used in the manufacturing of flavours and of dairy and other products do not result in predefined desired flavours since they lack or are deficient in certain enzymatic activities in the chain of converting amino acids into the flavoured component volatile compounds, at least to the necessary extent. By selecting a combination of two or more micro-organism strains as the mixed culture for the production of a product of choice, which together are capable of effecting all reactions necessary to convert the amino acids into said volatile products, products are obtained with enhanced or tailor-made flavour. The mixed cultures of the present invention, as defined above, are therefore believed to be new.

The mixed cultures according to the invention can be suitably used for the production of a variety of products including, for example, foodstuff, food ingredients, flavours, and others. When the product is a foodstuff or food ingredient, the micro-organisms that are comprised in the mixed culture should be food-grade. In a preferred embodiment of the invention the food product is a fermented food product, for example fermented sauces such as soy sauce and soybean milk, sausages, fermented vegetables such as cucumbers, sauerkraut and olives, baked goods such as fermented bread, marinated fish products, or, more preferably, a fermented dairy product, such as yogurt or, most preferably, cheese. In a particularly preferred embodiment of the invention, the mixed culture is a starter culture for the production of a dairy product, such as yogurt and, most preferably, cheese.

The micro-organisms which are comprised in the mixed cultures of the invention are selected from a wide range of suitable micro-organisms, depending on a number of factors, such as the product to be made and the desired flavour. In case the product is a dairy product, suitable micro-organism strains Include but are not restricted to strains of Lactococcus e.g. Lactococcus cremoris or lactis, Lactobacillus e.g. Lactobacillus helveticus, acidophilus or bulgaricus, Propionibacteria, Streptococcus e.g. Streptococcus thermophilus, Staphylococcus e.g. Staphylococcus aequorum, Bifidobacterium, Penicillium e.g. Penicillium camembertii or roquefortii, Brevibacterium e.g. Brevibacterium limens, Arthrobacter, Corynebacterium, Saccharomyces e.g. S. cerevisiae, Debaromyces e.g. D. hansenii, etc.

In addition, the mixed culture according to the invention may also comprise one or more further micro-organism strains or ingredients of choice which do not take part in the metabolic pathway to a desired flavour compound. For instance, a strain used for fast acidification of eye-formation.

In another aspect of the present invention a method is provided for the preparation of a mixed culture as defined above. The choice of the micro-organism strains which are comprised in the mixed culture is predominantly based on the putative pathway to the desired flavour end-product and/or the productivity of said flavour product. The properties of the micro-organism strains and the various techniques such as the use of the mixed cultures and/or the cultivation of the strains are known in the art and a skilled person will have no difficulty in making the proper selections based on the present description and his skill.

Suitable flavour compounds or components which can be formed using mixed cultures according to the present invention include, for example, branched-chain aldehydes, such as 3-methylbutanal, 2-methylbutanal and 2-methylpropanal, or derivatives thereof, which are obtainable from the branched-chain amino acids leucine, isoleucine and valine, respectively. Likewise, suitable flavouring aromatic aldehydes or derivatives thereof are obtainable from the corresponding aromatic amino acids phenylalanine and tyrosine. Also, suitable sulfur-containing flavour compounds, such as dimethyldisulfide and dimethyltrisulphide are obtainable from the amino acid methionine, through methanethiol.

In still another aspect of the present invention, the use of a mixed culture is provided, as herein defined, for the production of a variety of products, mentioned above, including, for example, foodstuff, food ingredients, drinks, health food, flavours, and others. A particularly preferred embodiment of this invention is the use of a mixed culture as a starter culture in the preparation of a dairy product, in particular cheese.

Growth and Flavour Production by Mixed Cultures

As a typical example of the present invention, using a mixed culture of strain B1157, a Lactococcus cremoris strain from artisanal origin, and a commercial Lactococcus cremoris strain, SK110, in milk resulted in a very strong chocolate-like flavour. Strain B1157 alone produces only a moderate chocolate-like flavour, whereas SK110 alone fails to produce this flavour. Headspace gas chromatography results corroborate the organoleptic evaluations. High levels of branched-chain aldehydes, which are indicative for a chocolate-like flavour, were found when B1157 and SK110 grew together.

Both strains appear to contain transaminase activity. Although, in addition, strain B1157 has a very high decarboxylating activity, the release of amino acids from milk protein is limited in this strain. On th other hand, strain SK110 is strongly limited in decarboxylating activity, whereas this strain is very active in proteolysis. Apparently, by combining these strains the substrates released by SK110 can be directly used by the other strain resulting in completion of the whole flavour-formation pathway.

In contrast a mixed culture of strain B851, another Lactococcus cremoris strain from artisanal origin, and strain SK110 did not result In a similar effect. This combination is therefore shown for comparison only. The two experiments will be described in some more detail below.

Lactococcus cremoris strains B1157 and B851 were grown individually in milk as well as in combination with the industrial Lactococcus cremoris strain SK110 and subsequently the milk cultures were organoleptically evaluated. See Table 2.

TABLE 2
Chocolate-like flavour score of milk cultures incubated with wild strains
B1157 and B851 and industrial strain SK110 (mean ± SD)

| Strain | Chocolate-like flavour^{a} |
|---|---|
| SK110 | 0 ± 0 |
| B1157 | 1.3 ± 0.5 |
| B851 | 1.9 ± 0.4 |
| B1157 + SK110 (2:1)^{b} | 2.9 ± 0.4 |
| B1157 + SK110 (1:2)^{b} | 1.8 ± 0.3 |
| B851 + SK110 (2:1)^{b} | 0.9 ± 0.6 |
| B851 + SK110 (1:2)^{b} | 0.7 ± 0.5 |

^{a}Scale from 0 (none) to 4 (very strong); results are mean values with standard deviations
^{b}Inoculation ratio

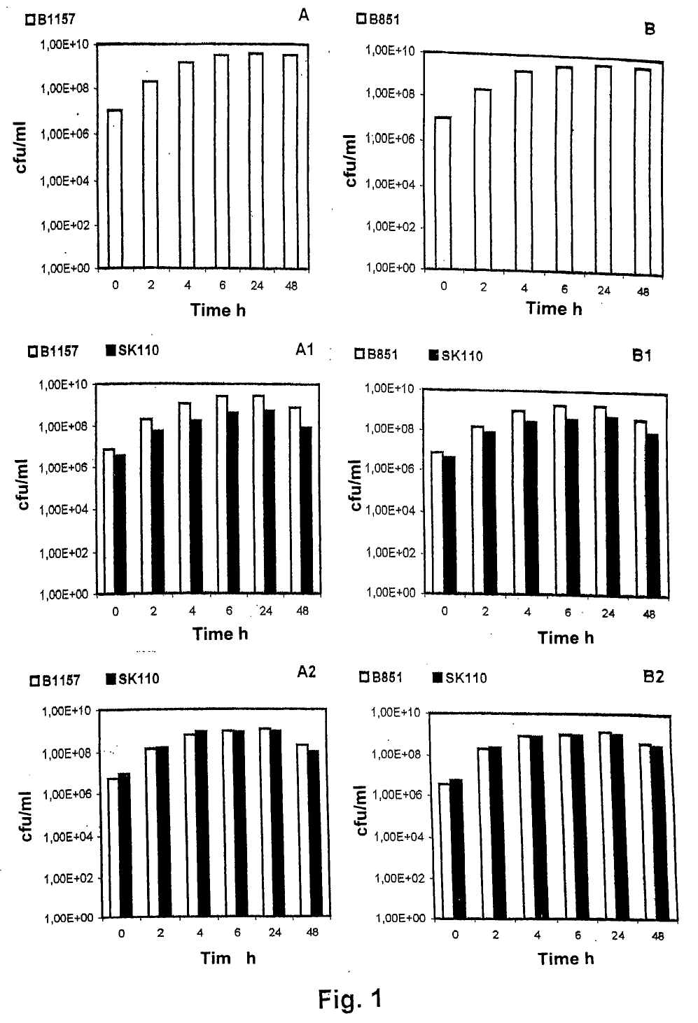

Strain B1157 produced a slight chocolate-like flavour in milk, when grown as a pure culture. Surprisingly, this flavour formation was significantly increased upon co-culturing with industrial strain SK110. This finding suggests that these cultures have a direct effect on each others metabolism. Such interactions are highly relevant for practical application. The growth of strains B1157 and B851, when cultured together with SK110 in two combinations (1:2 and 2:1) was followed by measuring the cell counts of the individual strains. Strains were distinguished individually based on proteolytic activity and the differences between growth temperature characteristics of lactococcal isolates from artisanal, non-dairy origins and industrial strains. The growth of individual and mixed cultures are shown in FIG. 1. Each strain can grow well both in a mixture as on its own. The initial balance between the strains remains stable during co-cultivation. Mixing at a ratio of 2:1 resulted in a higher intensity of the chocolate-like flavour than in case of the 1:2 ratio. Suitable ratios generally rang from 5:1 to 1:5, mainly depending on the specific properties aimed. Strain B851 produced a moderate chocolate-like flavour in milk when cultivated alone, whereas this flavour intensity was decreased when B851 was mixed with SK110 (Table 2). This reduction of chocolate-like flavour production is most likely due to the reduced number of B851 cells present in the mixed cultures, as compared to the situation in the individual cultures (FIG. 1).

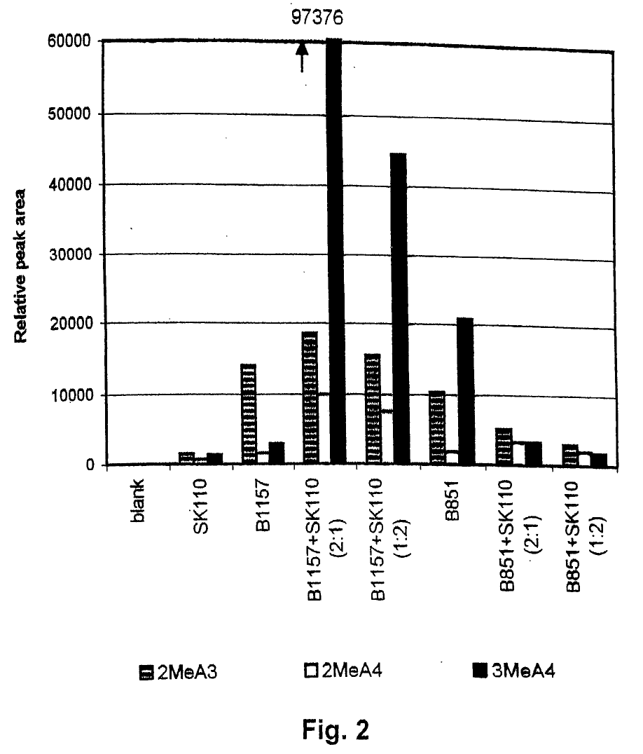

Considering the chocolate-like flavour that was perceived during the organoleptic evaluation, and the knowledge that branched-chain aldehydes derived from branched-chain amino acids can be responsible for the development of a malty flavour in milk and cheese (Morgan 1976; Dunn and Lindsay 1985; McDonald 1992; Barbieri et al. 1994; Urbach 1993), the milk culture samples were subjected to headspace gas chromatography (HS-GC). The conversion of leucine, isoleucine and valine proceeds via transamination of the amino acid to the corresponding α-keto acids and, subsequently, via a chemical or enzymatical decarboxylation step to 3-methylbutanal, 2-methylbutanal and 2-methylpropanal, respectively (Yvon et al. 1998; Christensen et al. 1999). The relative amounts of branched-chain aldehydes i.e. 3-methylbutanal (“3MeA4”), 2-methylbutanal (“2MeA4”) and 2-methylpropanal (“2MeA3”), formed during incubation of individual and mixed strains in milk cultures are presented in FIG. 2. Relatively high levels of particularly 3MeA4, but also 2MeA3 and 2MeA4 were found in the milk cultures incubated with B1157 and SK110. Much lower amounts of these aldehydes were detected in the milk culture incubated with B1157 alone, whereas these compounds were hardly present in the milk culture prepared with SK110 only. The amounts of aldehydes found in milk cultures incubated with mixtures of B851 and SK110 were lower than those encountered in milk incubated with B851 alone. The differences noticed in the amount of aldehydes, correspond with the organoleptic data.

The results indicate that in the combination of SK110 and B1157 a complete pathway for the formation of branched-chain aldehydes is actively present. Since the individual strains do not produce these aldehydes in high amounts, it is likely that this flavour formation is limited in each strain individually.

Conversion of Branched Chain Amino Acids by Lactococcal Enzymes

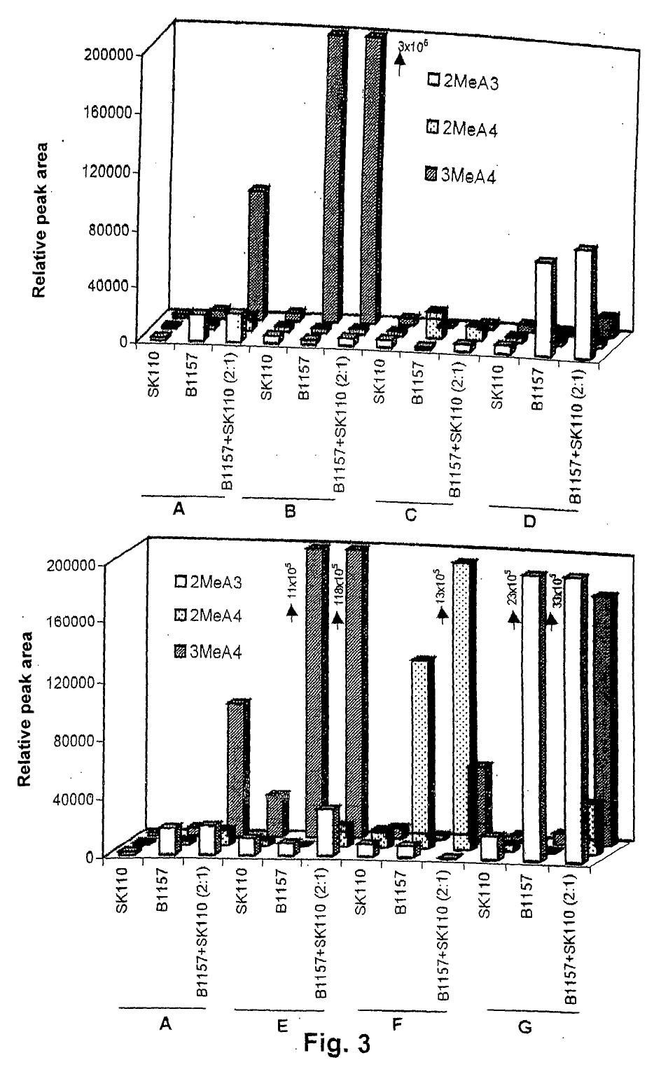

In order to obtain insight in the regulation of flavour formation in mixed cultures, the conversion routes of branched-chain amino acids into the corresponding aldehydes by SK110, B1157 and mixtures thereof were studied. The strains were incubated in milk either alone or together in a 2:1 ratio (SK110: B1157) In the absence or presence of leucine (Leu), isoleucin (Ile), or valin (Val) or their corresponding α-keto adds (α-keto isocaproic acid (KICA), α-keto-β-methyl-n-valeric acid or α-keto isovaleric acid, respectively). The volatile compounds (aldehydes) which were formed by enzymatic conversion were quantified using HS-GC (FIG. 3). Strain B1157 grown in milk contained a higher level of 2MeA3 and 3MeA4 than a culture of SK110, whereas the level of 2MeA4 was apparently similar to those in the culture of SK110. However, a milk culture prepared with a mixture of these strains (2:1) contained significantly higher levels of 2MeA3 and 3MeA4. These results indicate that strain B1157 is able to convert the branched-amino acids to the aldehydes, this conversion may be due to transamination followed by decarboxylation (Yvon et al. 1997; Yvon et al. 1998; Engels 1997; Christensen et al. 1999; Engels et al. 2000).

Addition of Leu to the milk cultures prepared with B1157 and mixtures of B1157 and SK110 resulted in an increase in the level of 3MeA4, whereas no effect was recorded for the culture of SK110 only. Addition of Ile to a culture containing B1157 resulted in an increase in the production of 2MeA4 and addition of Val led to an increase of 2MeA3. Addition of α-keto isocaproic acid, α-keto-β-methyl-n-valeric acid and α-keto isovaleric acid to pure and mixed cultures containing B1157 led to an increase in the corresponding aldehydes from each α-keto acid (FIG. 3). These results indicate that in the presence of high concentrations of the amino acids Leu, Ile, Val or their corresponding α-keto acids, transaminative dégradation of branched amino acids to their corresponding α-keto acids and the decarboxylation of the α-keto acids to the corresponding aldehydes can proceed in B1157 more extensively than in the absence thereof. The process of decarboxylation is also noticed after addition of α-keto acids to the milk. This reflects that the formation of amino acids seems to be the rate limiting step in aldehyde flavour production by this strain.

Free Amino Acid Analysis

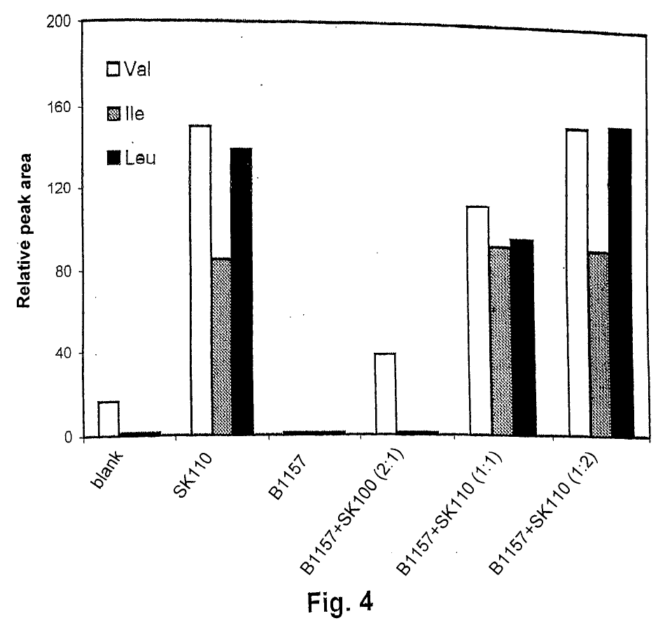

Free amino acid profiles of milk cultures incubated with SK110 and B1157 and their mixtures revealed that the amino acid patterns were different with SK110 from those with B1157 cultures due to the action of proteolytic enzymes (data not shown). SK110 is able to release the branched-chain amino acids (Leu, Ile and Val) whereas these amino acids were not liberated by B1157 cells (FIG. 4). Although SK110 is able to produce these amino acids, only low amounts of Val and neither Leu nor Ile were detected in the mixture of B1157 and SK110 at a ratio 2:1. This could be due to the direct conversion of these amino acids to branched-chain aldehydes by B1157. In the case of the mixtures 1:1 and 1:2 (B1157:SK110), branched-chain amino acids were present in the chromatogram. Apparently, when SK110 was present in equal or higher dose than B1157, amino acids converting enzymes became limiting. This results corroborates the difference in the organoleptic scores in chocolate intensity between the 2:1 and 1:2 (B1157:SK110) (Table 2).

Aminotransferase and Decarboxylase Activities

The aminotransferase activities towards leucine were determined in CFEs of B1157, B851, SK110 and two other L. lactis strains, B1173 and B850, from natural niches (for comparison). All strains showed aminotransferase-activity by the formation of KICA although some differences were observed. See Table 3.

TABLE 3
Relative amounts of α-keto isocaproic acid (“KICA”) and 3-methyl
butanal (“3MeA4”) formed by cell free extract (“CFE”) of L. lactis strains

Peak area

| CFE fraction | KICA¹ | 3MeA4² |
|---|---|---|
| Blank | 0.0 | 0.3 |
| SK110 | 84.5 | 0.4 |
| B1157 | 60.0 | 400 |
| B1173 | 36.0 | 93.3 |
| B850 | 119.0 | 54.9 |
| B851 | 136.0 | 48.0 |

¹Relative amounts of KICA as determined by reversed-phase of HPLC after incubation of CFE with leucin.
²Relative amounts of 3MeA4 determined by HS-GC after incubation of CFE with KICA (area expressed in arbitrary units)

CFE fractions inactivated by heat treatment gave no KICA formation (data not shown). These results indicate that all tested strains contain an active transaminase.

Decarboxylating activity towards KICA was measured in cell free extracts of the same strains (B1157, B1173, B850, B851 and SK110). The amount of 3MeA4 formed during incubation is indicative for a decarboxylating activity present in the CFE (Table 3). The amount of 3MeA4 formed from KICA in the presence of CFE from B1157 was the highest for all strains tested, indicating a high decarboxylating activity in this strain. No dégradation occurred in CFE from SK110 suggesting the absence of active decarboxylation by this strain. Heat inactivated CFE fractions gave no 3MeA4 formation (data not shown), which indicates that this conversion is enzymatic and the decarboxylation step was found to be specially “active” in wild-type strains. These lactococcal strains were previously found to be more dependent on their own synthesis of amino acids and, therefore, they probably possess more active amino acid convertases than the industrial strains (Ayad et al. 1999). As a consequence, the free amino acids will serve as flavour precursors and will be involved directly in the flavour production.

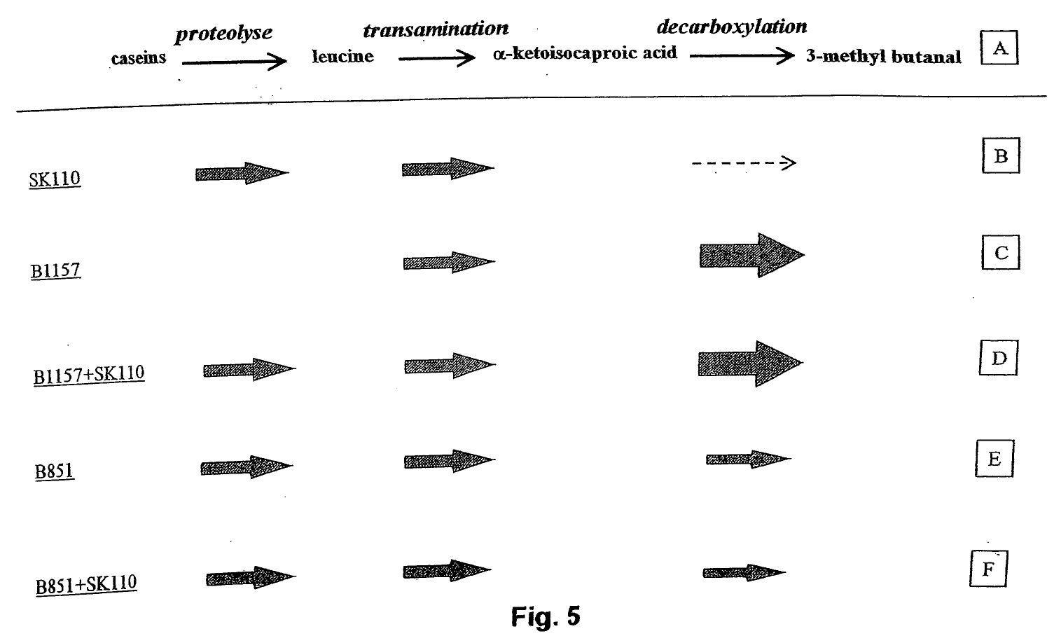

Taken together, the interaction between strains in the tested mixtures is schematically illustrated in FIG. 5. In SK110, the complete pathway from casein to 3-methyl butanal can not proceed due to the lack of a decarboxylative enzyme in this strain (FIG. 5B). B1157 is a non-proteolytic strain and therefore unable to produce enough free amino acids that can serve as a substrate for the subsequent transamination and decarboxylation steps (FIG. 5C). However, when B1157 and SK110 are incubated together, the strains complement each other with regard to their enzyme activities resulting in a high production of the chocolate flavour component 3-methyl butanal (FIG. 5D). On the other hand, strain B851 is able to carry out the whole dégradation (FIG. 5E), although its decarboxylase activity is lower than that of B1157. As a result, only a moderate chocolate-like flavour is found (FIG. 5F and Table 2). When B851 is mixed with SK110, the chocolate-like flavour intensity is experienced as being lower (Table 2). This might be due to a further “dilution” of enzyme activity in the mixture as compared to the pure culture of B851 (FIG. 1). Suggesting that, depending on the amount and the enzymes activity of such strains, the intensity of chocolate-like flavour can be controlled, when the branched-chain aldehydes compounds are in balance with other compounds, consequently, such flavour can be applied in a positive way. This could explain why these compounds have been recognized as off-flavours in raw milk (Morgan, 1976; Molimard and Spinnler, 1996) and also recognized as key flavours compounds in some artisanal cheeses (Bosset and Gauch, 1993; Barbieri et al., 1994; Neeter et al., 1996).

In conclusion, the amino acid converting enzymes of LAB can play an essential role in flavour development. In mixed cultures many different interactions can occur (Meers 1973), which not only affect the composition of these mixtures, but, as herein described, have an important impact on flavour production. The combination of knowledge of flavour formation pathways and functional characteristics of lactic acid bacteria cultures opens new avenues for industrial applications. It can be used inter alia to develop tailor-made starter cultures, as well as to produce flavour blocks.

A further typical example of the present invention is the use of specific mixes of starter cultures for surface-ripened (smear) cheese. For example, the flavour formation of such cultures can be enhanced using a combination of a Brevibacterium strain and a Staphylococcus strain as a starter culture. Smear-ripened cheeses, like Tilsit, Danbo, Limburger, and Appenzeller, host a relatively wide range of micro-organisms on their surface. The presence of Arthrobacter nicotianae, Brevibacterium linens, Brevibacterium casei, Micrococcus luteus, and Staphylococci on the surface of smear cheeses is well documented (Irlinger and Bergere, 1999). These micro-organisms, primarily bacteria and yeasts, are responsible for the production of various flavour compounds during ripening of the cheeses. We have now surprisingly found that co-cultivation of selected strains leads to the formation of significantly higher levels of key-flavour compounds as compared to the cultivation of individual strains, as exemplified by the following example.

In a pure culture of Brevibacterium casei B1392, the concentration of methanethiol was found to be higher than the background whereas no methanethiol was detected in cultures containing only Staphylococcus saprophyticus B1144 (Table 4). However, higher concentrations of methanethiol were formed in co-cultures containing Brevibacterium casei B1392 and S. saprophyticus B1144. These concentrations exceeded those in pure culture of these strains.

TABLE 4
Formation of methanethiol in cultures of Brevibacterium and
Staphylococcus

| Strain | Methanethiol (mg/l) |
|---|---|
| blanc | 0.01 |
| Staphylococcus saprophyticus B1144 | 0.01 |
| Brevibacterium casei B1392 | 0.09 |
| Brevibacterium casei NIZO B1392 and | 0.39 |
| Staphylococcus saprophyticus S1 | |

These results show that flavour formation during ripening of smear-cheeses can be optimised using the broad concept of the invention.

The invention will now be further illustrated by the following experimental work which, however, is not to be construed as limiting the scope of the present invention in any respect.

Materials and Methods

Chemicals

Amino acids (leucine, isoleudne and valine), α-keto adds (α-keto isocaproic acid (KICA), α-keto-β-methyl-n-valeric acid and α-keto-isovaleric acid) and thiamine pyrophosphate chloride (TPP) were obtained from Sigma Chemicals (St. Louis, Mo., USA), α-ketoglutaric acid was purchased from Janssen Chimica (Geel, Belgium), ethylenediaminetetra-acetc acid (EDTA) from BDH Limited (Poole, UK), and pyridoxal-5′-phosphate (PLP) from Boehringer Mannheim GmbH (Mannheim, Germany). All other chemicals used were of analytical grade.

Selection of Micro-Organisms

Natural flavours are usually obtained by the enzymatic activity of food-grade micro-organisms. A certain micro-organism strain contains a large part of the metabolic pathway leading from a substrate (e.g. an amino acid or an intermediary compound) to the end product, a specific type of flavour. Such an end product is for example an aldehyde, ketone, alcohol, ester, or sulfur compound. The way to select for these types of strains is to screen a micro-organism collection for flavour-producing enzyme activities by cultivating these micro-organisms, e.g. bacteria, in suitable media. Micro-organisms are then selected for each step of the pathway leading to a certain flavour (compound). For each step, a screening for specific enzymatic activities is required. In the literature various methods for such screening activities are reported. In the example given, the pathway necessary to obtain the aldehyde with a strong flavour impact, a combination of strains is presented in which one strains supplies free amino acids to the other strains which is able to convert the substrate (leucine) via a two-step pathway into 3-methyl butanal. The two-step pathway consists of an aminotransferase activity on leucine and a decarboxylase activity on the α-ketoacid released by the aminotransferase.

The next step is to cultivate the combination of the selected bacteria in the presence of the right substrate in order to have a complete pathway present. It was surprisingly found in accordance with the present invention that it is not required to have a whole pathway present in one bacterial strain, since apparently the strains are able to exchange intermediate compounds of the selected pathway.

Micro-Organisms and Growth Conditions

The following strains were used: (i) strain Lactococcus lactis subsp. cremoris SK110 (NIZO B697), which is derived from a commercial starter culture, (ii) the strains L. lactis subsp. cremoris NIZO B1157, L. lactis subsp. lactis NIZO B851, L. lactis subsp. lactis NIZO B850 and L. lactis subsp. lactis NIZO B1173, which originate from natural niches (Ayad et al. 1999). The Lactococcus strains were routinely stored in litmus milk with CaCO₃ and 0.5% yeast extract and kept at −40° C. Strains B1157 and B1173 are non-proteolytic strains, which were grown in milk with 0.5% yeast extract, whereas SK110, B850 and B851 are proteolytic strains, which were cultured in milk without yeast extract.

The Brevibacterium and Staphylococcus strains were precultured in DNB (Difco Nutrient Broth) for 96 h (Brevibacterium) or overnight (Staphylococcus). Aliquots of these cultures were added to fresh DNB medium in headspace vials to an optical density at 600 nm of 0.02 for Brevibacterium or 0.01 for Staphylococcus. These cultures were incubated for three days and analysed by GC using a headspace autosampler. Concentrations were calculated using appropriate calibration curves.

Lactococcus cremoris strain SK110 is a commercially available strain which can be purchased from Nizo food research.

Lactococcus cremoris strain B1157 was deposited on Jun. 20, 2000 with the Centraal Bureau voor Schimmelcultures, in Baam, the Netherlands, under CBS 108917.

Lactococcus cremoris strains B850, B851, and B1173 were used herein for comparison only and can be obtained on request from Nizo food research.

Brevibacterium casei strain B1392 was deposited on Jun. 29, 2001 with the Centraal Bureau voor Schimmelcultures, Baam, the Netherlands, under CBS 109543.

Staphylococcus saprophyticus strain B1144 was deposited on Jun. 29, 2001 with the Centraal Bureau voor Schimmelcultures, Baam, the Netherlands, under CBS 109544.

Flavour Production and Population Dynamics

Individual strains, SK110, B1157, B851 were pre-cultured for 16 h at 30° C. in sterilised milk with 0.5% yeast extract for non-proteolytic strain and without yeast extract for proteolytic strains. Cultures consisting of a strain isolated from natural niches were combined with cultures of the industrial strain (SK110) in different ratios (2:1 and 1:2) at a final total inoculum level of 1% (v/v) and grown together in 500 ml skimmed UHT milk for 48 h at 30° C. The strains were also inoculated individually at 1% and grown under the same conditions.

The total number of cells (colony-forming units) in each milk culture was determined by plating cells on GMA agar containing 10% skimmed milk, 1.9% β-glycero-phosphate (pH 6.9), 0.001% bromocresolpurple and 1.3% agar as described previously (Limsowtin and Terzaghi 1976; Hugenholtz et al. 1987). Based on the differences in the ability to hydrolyse casein and the ability to grow at 40° C. between wild-type strains and the industrial strain (Ayad et al 2000), the cell number of the individual strains could be monitored in a mixed population.

The milk cultures were sensorically evaluated by 5-8 experienced cheese graders. The attributes were recorded and statistically analysed. The flavour intensity scale ranged from 0 [none] to 4 [very strong].

Analysis of Volatile Compounds

Branched aldehydes formed by the cultures used, were Identified and quantified using headspace gas chromatography (HS-GC). The analytical system used consisted of a headspace autosampler HS800 mounted on a Mega series gas chromatograph (CE instruments, Thermo Quest, Milan, Italy) fitted with a splitless injector, a flame ionisation detector and a fused silica capillary column (25 m×0.22 mm i.d., d_{f}=1 μm CP-Sil5 CB-LB, Chrompack, the Netherlands). After an equilibration time of 20 min at 60° C., headspace samples (1.0 ml) were injected directly (splitless) onto a capillary pre-column (25 cm×0.53 mm). The latter was mounted in a cryotrap model 515 (Thermo Quest, Milan, Italy) inside the oven. During Injection the volatile compounds are condensed (−150° C.) and adsorbed in this capillary pre-column and then re-injected onto the chromatographic column by flash heating (150° C.). GC separation was performed under isothermal conditions (70° C.) at a carrier gas flow rate of 1.2 ml/min hydrogen. Identification of aldehydes was achieved using retention times of standard compounds.

Enzymatic Conversion of Branched Chain Amino Acids by Strains of L. lactis

Cultures were pre-grown in sterilised milk, containing 0.5% yeast extract for non-proteolytic strains, overnight at 30° C., and subsequently, individual and mixed cultures (B1175+SKI10 2:1) were grown in 50 ml UHT milk after inoculation at a final inoculum level of 1% (v/v). The following additions were made: 1) no additions; 2) 10 mM leucine; 3) 10 mM isoleucine; 4) 10 mM valine; 5) 10 mM α-ketolsocaproic acid; 6) 10 mM α-keto-β-methyl-n-valeric acid and 7) 10 mM α-ketoisovaleric acid. The volatile components formed enzymatically by the strains were detected by using direct static headspace injection in combination with gas chromatography and flame ionisation detection. Column and chromatographic conditions were the same as those described above.

Free Amino Acid Analysis

Free amino acids were determined on a 4151 Alpha Plus amino acid analyser (Pharmacia LKB, Uppsala, Sweden). The soluble nitrogen fractions (Noomen 1977) were prepared from skimmed UHT milk incubated with individual strains SK110 and B1157 and their mixtures in different ratios at final Inoculum level of 1% for 48 h at 30° C.

Preparation of Cell-Free Extract (CFE)

The strains were cultured overnight at 30° C. in sterilised milk with 0.5% yeast extract only for non-proteolytic strains. After addition of 1% (w/v) sodium tricitrate, the cells were harvested by centrifugation (5 min, 10000 g, 4° C.) and washed twice in 50 mM potassium phosphate buffer (pH 7.5). The washed cells were resuspended to an OD₆₀₀ₙₘ of approximately 20 (Ultrospec 3000, Pharmacia Biotech., UK) in the same buffer, added to a plastic tube (Sarstedt 72694) with 1 gram glass beads (Zirconium beads Ø0=0.1 mm) and kept on ice (0° C.). The cells were disrupted by using a Bead beater (multipurpose Orbital mixer) for 3×3 min, cooled on ice for 2 min after every 3 min of shaking. The treated suspension was centrifuged (3 min, 14000 g, 4° C.) to remove intact bacteria and cell debris, and the supernatant (CFE) was collected. CFE was stored at −30° C. until further use.

Determination of Aminotransferase and Decarboxylase Activity

The aminotransferase activity in CFE of wild strains and the industrial strain SK110 was measured as follows: 100 μl of CFE (either active or inactive by heat treatment) was incubated in 20 mM potassium phosphate buffer (pH 7.5) containing 1 mM EDTA and 20 μM PLP, with leucine (final concentration 20 mM) and co-substrate α-ketoglutaric acid (final concentration 10 mM). The final volume of the incubation mixture was 200 μl. The incubations were performed at 30° C. for 1 h in the dark. The reaction was stopped by lowering the pH of the mixture to 2.5 via addition of 0.2 M HC1. The formation of α-keto isocaproic acid (KICA) during incubation was quantified by measuring its peak area using high-performance liquid chromatography (HPLC). The HPLC equipment used was as described before (Engels 1997). The relative amounts of KICA were determined from their peak area. Perkin Elmer. Nelson Turbochrom 4.0 software (Cupertino, Calif.) was used for processing raw HPLC data.

The conversion of KICA to 3-methyl butanal (3MeA4) by CFE was monitored by determining 3MeA4 using headspace gas chromatography with flame-ionisation detection (see above). CFE (100 μl) either active or Inactive by heat treatment was incubated in 50 mM potassium phosphate buffer (pH 6.0) at 35° C. for 4 h containing 1 mM EDTA, 50 μM TPP, and KICA (final concentration 5 mM). The reaction was stopped by adding 50 μl of 6 M (HCl) to reduce the pH to 2.

### R F RENCES

Alting, A. C., Engels, W. J. M., Van Schalkwijk, S. and Exterkate, FA (1995) Purification and characterization of cystathionine/3 lyase from Lactococcus lactis subsp. cremoris B78 and its possible role in flavor development in cheese. Applied and Environmental Microbiology 61,4037-4042.

Ayad, E. H. E., Verheul, A., De Jong, C., Wouters, J. T. M. and Smit, G. (1999) Flavour forming abilities and amino acid requirements of Lactococcus lactis strains isolated from artisanal and non-dairy origin. International Dairy Journal 9, 725-735.

Barbieri, G., Bolzoni, I., Careri, M., Manglia, A., Parolari, G., Spagonoli, S. and Virgili, R. (1994) Study of the volatile fraction of parmesan cheese. Journal of Agricultural and Food Chemistry 42, 1170-1176.

Berger, R. G. (1992) Naturally-occurring flavours from fungi, yeast, and bacteria. In: Bioformation of flavours, eds Patterson, R. L. S., Char;wood, B. V., MacLeod, G. and Williams, A. A., Royal Society of Chemistry, UK.

Bie, R., and Sjostrom, G.(1975a) Autolytic properties of some lactic acid bacteria used in cheese production. Part I: Material and methods. Milchwissenschaft 30, 653-657.

Bie, R., and Sjostrom, G.(1975b) Autolytic properties of some lactic acid bacteria used in cheese production. Part II: Experiments with fluid substrates and cheese. Milchwissenschaft 30: 739-747.

Bosset, J. O. and Gauch, G. (1993) Comparison of the volatile flavour compounds of six European ‘AOC’ cheeses using a new dynamic headspace GC-MS method. International Dairy Journal 3, 423-460.

Christensen, J. E., Dudley, E. G., Pederson, J. A., and Steele, L. J. (1999) Peptidases and amino acid catabolism in lactic acid bacteria. Antonie van Leeuwenhoek 76:217-246.

Dunn, H. C. and Lindsay, R. C. (1985) Evaluation of the role of microbial Strecker-derived aroma compounds in unclean-type flavours of Cheddar cheese. Journal of Dairy Science 68: 2859-2874.

Engels, W. J. M. (1997) Volatile and non-volatile compounds in ripened cheese: their formation and their cotribution to flavour. PhD thesis, Wageningen Agricultural University, Wageningen, the Netherlands.

Engels, W. J. M. and Visser, S. (1994) Isolation and comparative characterization of components that contribute to the flavour of different types of cheese. Netherlands Milk and Dairy Journal 48,127-140.

Engels, W. J. M., and Visser, S. (1996) Development of cheese flavour from peptides and amino acids by cell-free extracts of Lactococcus lactis subsp. cremoris B78 in a model system. Netherlands Milk and Dairy Journal 50, 3-17.

Engels, W. J. M., Alting, A. C., Arntz, M. M. T. G., Gruppen, H., Voragen, A. G. J., Smit, G. and Visser, S. (2000) Conversion of methionine and branched-chain amino acids by Lactococcus lactis subsp. Cremoris: purification and partial characterization of branched-chain aminotransferases involved. Submitted for publication.

Exterkate, F. A. and Alting, A. C. (1995) The role of starter peptidases in the initial proteolytic events leading to amino acids in Gouda cheese. International Dairy Journal 5, 15-28.

Exterkate, F. A., Alting, A. C. and Slangen, C. J. (1995) Conversion of αₐ₁-casein-(24-199) fragment and β-casein under cheese conditions by chymosin and starter peptidase system. Applied Microbiology 18,7-12.

Gao, S., Oh, D. H. and Steele, J. L. (1997) Aromatic amino acid catabolism by lactococci. Lait 77,371-381.

Gao, S. and Steele, J. (1998). Purification and characterization of oligomeric species of an aromatic amino acid aminotransferase from Lactococcus lactis subsp. lactis S3. Journal of Food Biochemistry 22, 197-211.

Hemme, D., Bouillanne, C., Métro, F. and Desmazeaud, M.-J. (1982) Microbial catabolism of amino acids during cheese ripening. Sci. Alim. 2:113-123.

Hugenholtz, J, Splint, R., Konings, W. N. and Veldkamp, H. (1987) Selection of proteinase-positive and proteinase-negative variants of Streptococcus cremoris. Applied and Environmental Microbiology 63, 309-314.

Irlinger, F., Bergere, J. L., Journal of Dairy Research 66(1):91-103 (1999).

Law, B. A., Sharpe, M. E. and Reiter, B. (1974) The release of intracellular dipeptidase from starter streptococci during Cheddar cheese ripening. Journal of Dairy Research 41, 137-146.

Law, B. A. and Mulholland, F. (1995) Enzymology of lactococci in relation to flavour development from milk proteins. International Dairy Journal 5, 833-854.

Lee, C. W. and Richard, J. (1984) Catabolism of phenylalanine by some micro-organisms of cheese origin. Journal of Dairy Research 51, 461469.

Lee, C. W., Lucas, S. and Desmazeaud, M. J. (1985) Phenylalanine and tyrosine catabolism in some cheese coryneform bacteria. FEMS Microbiology Letters 26, 201-205.

Limsowtin, G. and Terzaghi, B. E. (1976) Agar medium for the differentiation of “fast” and “slow” coagulating cells in lactic streptococcal cultures. New Zealand Journal of Dairy Science and Technology 11,65-66.

McDonald, S. T. (1992) Role of alpha-dicarbonyl compounds produced by lactic acid bacteria on the flavor and color of cheese. PhD thesis, University of Wisconsin, Madison, U.S.A.

Meers, J. L. (1973). Growth of bacteria in mixed cultures. Critical Reviews in Microbiology 2,139-184.

Molimard, P. and Spinnier, H. (1996) Compounds involved in the flavour of surface mold-ripened cheese: origins and properties. Journal of Dairy Science 79, 169-184.

Morgan, M. E. (1976) The chemistry of some microbially induced flavour defects in milk and dairy foods. Biotechnology and Bioengineering 18, 953-965.

Mulder, H. (1952) Taste and flavour forming substances in cheese. Netherlands Milk and Dairy Journal 6,157-168.

Nakazawa, H., Sano, K., Kumagai, H. and Yamada, H. (1977) Distribution and formation of aromatic L-amino acid decarboxylase in bacteria. Agriculture Biological Chemistry 41,2241-2247.

Neeter, R., De Jong, C., Teisman, H. G. J. and Ellen, G. (1996) Determination of volatile components in cheese using dynamic headspace techniques. In A. J. Taylor & Mottram Eds, Flavour science: Recent developments. Royal Society of Chemistry (Burlington House, London), p. 293-296.

Noomen, A. (1977) Noordhollandse Meshanger cheese: a model for research on cheese ripening. 2. The ripening of the cheese. Netherlands Milk and Dairy Journal 31, 75-102.

Olson, N. F. (1990) The impact of lactic acid bacteria on cheese flavor. FEMS Microbiology Reviews 87,131-148.

Pritchard, G. G. and Coolbear, T. (1993) The physiology and biochemistry of proteolytic system in lactic acid bacteria. FEMS Microbiology Reviews 12, 179-206.

Roudot-Algaron, F. and Yvon, M. (1998). Aromatic and branched-chain amino acid catabolism in Lactococcus lactis. Lait 78, 23-30.

Rijnen, L., Bonneau, S. and Yvon, M. (1999) Genetic characterization of the major aromatic aminotransferase and its involvement in conversion of amino acids to aroma compounds. Appl. Environ. Microbiol. 65:4873-4880.

Schmidt, J. L. and Lenoir, J. (1974) Contribution à l'étude des entérocoques et de leurs aptitudes technologiqu s. Aptitude à la dégradation des acides amines. Lait 54, 359-385.

Smit, G., Verheul, A., Kranenburg, R., Ayad, E., Siezen, R. and Engels, W. (2000) Cheese flavour development by enzymatic conversions of peptides and amino acids. Journal of Food Research International. In press

Solms, J. (1969) The taste of amino acids, peptides and proteins. Journal of Agricultural and Food Chemistry 17, 686-688.

Thirouin, S., Rijnen, L., Gripon, I.-C. and Yvon, M. (1995) Inventaire des activités de dégradation des acides amines aromatiques et des acides aminés à chaines ramifièes chez Lactococcus lactis, abstr. M4 Club des bacteries lactiques-7ème Colloque, Paris, France.

Visser, S. (1993) Proteolytic enzymes and their relation to cheese ripening and flavor an overview. Journal of Dairy Science 76, 329-350.

Urbach, G. (1993) Relations between cheese flavour and chemical composition. International Dairy Journal 3, 389422.

Yvon, M., Thirouin, S., Rijnen, L., Fromentier, D. and Gripon, J. C. (1997) An aminotransferase from Lactococcus lactis initiates conversion of amino acids to cheese flavor compounds. Applied and Environmental Microbiology 63, 414419.

Yvon, M., Berthelot, S. and Gripon, I. C. (1998) Adding a.-ketoglutarate to semi-hard cheese curd highly enhances the conversion of amino acids to aroma compounds. International Dairy Journal 8, 889-898.

1. A mixed culture of two or more micro-organism strains, characterised in that at least one of said micro-organism strains comprised in said mixed culture is individually selected on the basis of its ability to perform part of an enzymatic pathway, and said two or more micro-organism strains together form a complete pathway towards a desired flavour compound, which is a culture for use in the manufacture of fermented products with enhanced or tailor-made flavour formation, wherein said at least one of said micro-organism strains is individually less capable or incapable of converting amino acids into volatile compounds to the extent of forming the desired flavour, but when used together convert one or more amino acids into volatile compounds under appropriate conditions giving the desired flavour.

2. A mixed culture as claimed in claim 1, which is a culture for use in the manufacture of dairy products with enhanced or tailor-made flavour formation, wherein said at least one of said micro-organism strains is individually less capable or incapable of converting amino acids into volatile compounds to the extent of forming the desired flavour, but when used together convert one or more amino acids into volatile compounds under appropriate conditions giving the desired flavour.

3. A mixed culture as claimed in claim 1 or 2, further comprising one or more further micro-organism strains or ingredients of choice which do not take part in the metabolic pathway to said desired flavour compound.

4. A mixed culture as claimed in any one of claims 1 to 3, wherein said at least two or more micro-organism strains are co-cultivated.

5. A mixed culture as claimed in any one of claims 1 to 4, wherein said at least two or more micro-organism strains are selected from the group consisting of Lactococcus, Lactobacillus, Propionibacteria, Streptococcus, Staphylococcus, Bifidobacterium, Penicillium, Brevibacterium, Arthrobacter, Corynebacterium, Saccharomyces, and Debaromyces.

6. A mixed culture as claimed in claim 5, wherein said at least two or more micro-organisms are selected from the genus of Lactococcus strains.

7. A mixed culture as claimed in claim 6, wherein said at least two or more micro-organism strains comprise Lectococcus strain B1157 and Lectococcus strain SK110.

8. A mixed culture as claimed in claim 7, wherein the ratio of said Lactocoaccus strain B11157 and said Lactococcus strain SK110 ranges from about 1:5 to about 5:1.

9. A mixed culture as claimed in claim 5, wherein said at least two or more micro-organisms are selected from the group consisting of Brevibacterium, in particular Brevibacterium casei, and Staphylococcus, in particular Staphylococcus saprophyticus.

10. A mixed culture as claimed in claim 9, wherein said at least part of said micro-organism strains comprise Brevibacterium casei strain 81392 and Staphylococcus saprophyticus strain B1144.

11. A method for the preparation of a mixed culture, as defined in any one of claims 1 to 10, which comprises the following steps:
selecting two or more micro-organism strains which are individually less capable or incapable of converting one or more acids into volatile compounds to the extent of forming a desired flavour, but when used together convert said one or more amino acids into volatile compounds under appropriate conditions giving the desired flavour, and
optionally adding one or more further micro-organism strains or ingredients of choice which do not take part in the metabolic pathway to said desired flavour compound.

12. Use of a mixed culture, as defined in any one of claims 1-10, for the production of an entity selected from the group of foodstuff, food Ingredients, and flavour compounds.
